(19) United States
(12) Patent Application Publication
(10) Pub. No.: US 2012/0039814 A1
(43) Pub. Date: Feb. 16, 2012

(54) Topical Compositions and Methods of Detection and Treatment

(76) Inventors: Jennifer L. Sample, Chevy Chase, MD (US); Julia B. Patrone, Ellicott City, MD (US); Jason J. Benkoski, Ellicott City, MD (US); James C. Crookston, Falls Church, VA (US); Huong Le, Olney, MD (US); Jennifer L. Breidenich, Atlanta, GA (US); Lisa A. Kelly, Ellicott City, MD (US)

(21) Appl. No.: 13/208,874

(22) Filed: Aug. 12, 2011

Related U.S. Application Data
(60) Provisional application No. 61/373,327, filed on Aug. 13, 2010.

(51) Int. Cl. A61K 49/00; A61P 43/00; A61K 9/14

(52) U.S. Cl. 424/9.8; 424/498

(57) ABSTRACT

A topical composition includes a nanoemulsion of a plurality of hydrophobic particles having a hydrophilic coating therein. The hydrophobic particles are derived from the same or different hydrophobic material and each hydrophobic particle has a melting point below the melting point of the respective hydrophobic material. The nanoemulsion further includes one or more pharmaceutically active agents and/or one or more chemiluminescent disease-detecting systems.

### CROSS-REFERENCE TO RELATED APPLICATIONS

This application claims the benefit of prior filed co-pending U.S. Provisional Application No. 61/373,327, filed Aug. 13, 2010, the contents of which are herein incorporated by reference in their entirety.

### BACKGROUND OF THE INVENTION

1. Field of the Invention

The present invention generally relates to topical compositions and methods of detection and/or treatment.

2. Description of the Related Art

Nanotechnology is becoming increasingly more important in the pharmaceutical, chemical and engineering fields. This is primarily due to the fact that particles made at the nanoscale have much different physical, chemical, and biological properties than larger particles. For example, in the pharmaceutical field, nanoparticles have been used to more efficiently deliver drugs, genes, diagnostics, and vaccines. Due to their small size, nanoparticles can aid in the direct entry of entrapped molecules into, for example, cells.

There is a great variety of known liquid or semisolid preparations for topical application to the body, and they are generally based on aqueous or other polar liquids, on liquid or semisolid lipids, or on mixtures thereof. When the preparation is based on a mixture of an aqueous liquid and a lipid substance, the preparation is an emulsion, which may be a water-in-oil (w/o) emulsion in which the lipid substance is the continuous phase, or an oil-in water (o/w) emulsion in which the aqueous liquid is the continuous phase. Each of these types of emulsions is prepared with its own type of emulsifier. Examples of known emulsions of the w/o type are the ointments, which generally are semisolid. Examples of known emulsions of the o/w type are lotions, which are liquid, and creams and gels, which are semisolid.

The liquid or semisolid lipids contained in the liquid or semisolid water-in-oil (w/o) or oil-in-water (o/w) emulsions generally are responsible for a cosmetically and medically important effect, i.e., occlusion. By occlusion is meant the formation of a “barrier”, which causes reduction of water loss through the epidermis, after treatment thereof with such lipid containing emulsions. The occlusive effect is positively correlated to the lipid content of the emulsion. The resulting desirable cosmetic effect of occlusion is emolliency. The resulting desirable medical effect of occlusion is a better penetration into the skin and a better effectiveness of many medicaments, incorporated into an occluding emulsion, after topical application of the same. On the other hand, such lipid containing emulsions have the disadvantage that they are greasy and messy, resulting in a shining appearance of the treated area and in staining of the clothes, and these undesirable properties are also correlated to the lipid content of the emulsion.

U.S. Pat. No. 5,576,016 discloses a pharmaceutical composition containing nanoemulsions of particles comprising a lipid core which is in a solid or liquid crystalline phase at 25° C., stabilized by at least one phospholipid envelope, for the parenteral, oral, intranasal, rectal, or topical delivery of both fat-soluble and water-soluble drugs.

U.S. Pat. No. 5,904,932 discloses topical compositions containing an aqueous suspension of solid lipoid nanoparticles, containing at least one lipid and at least one emulsifier.

There remains a need for an improved topical composition containing a nanoemulsion of a plurality of lipid particles that can penetrate into the skin for therapeutic use and/or disease detection.

### SUMMARY OF THE INVENTION

In accordance with one embodiment of the present invention, there is provided a topical composition comprising a nanoemulsion of a plurality of hydrophobic particles having a hydrophilic coating therein, wherein the hydrophobic particles are derived from the same or different hydrophobic material and each hydrophobic particle has a melting point below the melting point of the respective hydrophobic material, and further wherein the nanoemulsion further includes one or more pharmaceutically active agents and/or one or more chemiluminescent disease-detecting systems.

In accordance with a second embodiment of the present invention, there is provided a method for detecting a disease or a condition in need of treatment, the method comprising (a) contacting an area of a host with an effective amount of a topical composition comprising a nanoemulsion of a plurality of hydrophobic particles having a hydrophilic coating therein, wherein the hydrophobic particles are derived from the same or different hydrophobic material and has a melting point below the melting point of the respective hydrophobic material, and further wherein the nanoemulsion further includes one or more chemiluminescent disease-detecting systems; and (b) determining if the one or more chemiluminescent disease-detecting system emits a detectable chemiluminescence thereby indicating the presence of a disease or a condition.

In accordance with a third embodiment of the present invention, there is provided a method for treating a host for a disease condition, the method comprising topically applying an effective amount of a topical composition to the host; wherein the topical composition comprises a nanoemulsion of a plurality of hydrophobic particles having a hydrophilic coating therein, wherein the hydrophobic particles are derived from the same or different hydrophobic material and each hydrophobic particle has a melting point below the melting point of the respective hydrophobic material, and further wherein the nanoemulsion includes one or more pharmaceutically active agents.

In accordance with a fourth embodiment of the present invention, there is provided a test kit for detecting a disease or a condition in a host, the kit comprising (a) a source of a topical composition comprising a nanoemulsion of a plurality of hydrophobic particles having a hydrophilic coating therein, wherein the hydrophobic particles are derived from the same or different hydrophobic material and has a melting point below the melting point of the respective hydrophobic material, and further wherein the nanoemulsion further includes one or more chemiluminescent disease-detecting systems; and (b) a light emitting detector for detecting a disease or a condition in the host.

By containing one or more chemiluminescent disease-detecting systems, the topical compositions of the present invention can advantageously detect one or more diseases or conditions in need of treatment in a host. In addition, the topical compositions of the present invention can also treat a host for a disease condition such as a skin condition.

### BRIEF DESCRIPTION OF THE DRAWINGS

FIGS. 1 and 2 show a chemical reaction that produces chemiluminescence.

FIG. 3 is a graph showing the chemiluminescence intensity versus time for a nanoemulsion of one embodiment of the present invention.

### DETAILED DESCRIPTION

The term “treat” or “treating” or “treatment” of a state, disorder or condition as used herein means: (1) preventing or delaying the appearance of clinical symptoms of a state, disorder or condition developing in a host that may be afflicted with or predisposed to the state, disorder or condition but does not yet experience or display clinical or subclinical symptoms of the state, disorder or condition, (2) inhibiting the state, disorder or condition, i.e., arresting or reducing the development of the disease or at least one clinical or subclinical symptom thereof, or (3) relieving the disease, i.e., causing regression of the state, disorder or condition or at least one of its clinical or subclinical symptoms i.e., prevented from happening, or stopped, e.g., terminated, such that the host no longer suffers from the state, disorder or condition, or at least the symptoms that characterize the state, disorder or condition. As such, treatment includes both curing and managing a disease condition.

The term “therapeutically effective amount” as used herein means the amount of a compound that, when administered to a subject for treating a state, disorder, condition or causing an action is sufficient to effect such treatment or action. The “therapeutically effective amount” will vary depending on the compound, the disease and its severity and the age, weight, physical condition and responsiveness of the mammal to be treated.

The term “delivering” as used herein means providing a therapeutically effective amount of one or more pharmaceutically active agents to a particular location within a host means causing a therapeutically effective blood concentration of the one or more pharmaceutically active agents at the particular location. This can be accomplished, e.g., by topical, local or by systemic administration of the one or more pharmaceutically active agents to the host.

The term “subject” or “patient” or “host” as used herein refers to mammalian animals, preferably human.

The term “topically” as used herein refers to application of the compositions of the present invention to the surface or subsurface of the skin and mucosal cells and tissues (e.g., alveolar, buccal, lingual, masticatory, or nasal mucosa, and other tissues and cells which line hollow organs or body cavities).

The present invention is directed to a topical composition comprising a nanoemulsion of a plurality of hydrophobic particles having a hydrophilic coating therein, wherein the hydrophobic particles are derived from the same or different hydrophobic material and each hydrophobic particle has a melting point below the melting point of the respective hydrophobic material, and further wherein the nanoemulsion further includes one or more pharmaceutically active agents and/or one or more chemiluminescent disease-detecting systems.

Hydrophobic Particles

Suitable hydrophobic materials for use in forming the hydrophobic particles include any hydrophobic material having a melting point of no more than about 85° C. In one embodiment, suitable hydrophobic material includes those having a melting point of no more than about 30° C. In one embodiment, suitable hydrophobic material include those having a melting point of from about 25° C. to about 37° C. Representative examples of such hydrophobic material include waxes and butters, oily substances with melting points above or near room temperature, including Avocado Butter, Almond Butter, Beeswax, White, Candellila Wax, Coffee Butter, Cocoa Butter, Mango Butter, Mowrah Butter, Palm Kernel Flakes, and Pistachio Butter. Useful waxes include, but are not limited to, paraffin wax, beeswax white, candellila wax, and the like and mixtures thereof. In one embodiment, the butter comprises vegetable butters. Vegetable butters can be created by blending plant extracts with fatty fractions of the same or different plant. One such example is aloe butter, which comprises aloe extract and cocoa butter. Vegetable butters can also be obtained by blending the fatty fractions of a vegetable oil. Fatty fractions can be combined with other fatty fractions from the same or different plant source. An example of fatty fraction is an unsaponifiable fraction containing paraffin, tocopherols and sterols. The percentage of the unsaponifiable fraction of a vegetable oil is usually very low and thus, unsaponifiable fractions are sometimes blended with refined vegetable oils that have undergone hydrogenation. Generally, it requires a large quantity of processed oil to yield a significant quantity of butter. Vegetable butters generally have a high content of symmetrical triglyceride comprising saturated and monounsaturated fatty acids, in particular stearic acid and oleic acid.

Useful butters include, but are not limited to, mangifera indica (mango) seed butter, aloe butter, olea europa (olive) butter, coffea arabica (coffee) bean butter, macadamia nut butter, persea Gratissima (avocado) butter, theobroma cacao (cocoa) seed butter, hemp seed butter, shorea stenoptera (illipe) seed butter, garcinia indica (kokum) seed butter, pistachio nut butter, butyrospermum parkii (shea butter), prunus amygdalus dulcis (sweet almond) butter, grape seed butter, bassia latifolia (mowrah) butter, prunus armeniaca (apricot) butter, shorea robusta (sal) butter, glycine soja (soy) butter, triticum vulgare (wheat germ) butter, palm kernel flakes and the like and mixtures thereof.

Suitable hydrophilic material for use in providing a hydrophilic coating on the hydrophobic particles include any hydrophilic material known in the art. Representative examples of hydrophilic material include anionic surfactants, cationic surfactants, amphoteric surfactants and non-ionic surfactants and the like, and mixtures of the foregoing.

The hydrophobic particles derived from the same or different foregoing hydrophobic material will have a melting point below the melting point of the respective hydrophobic material. As discussed hereinbelow, the process for preparing the nanoemulsion involves adding the hydrophobic material (also referred to as the bulk hydrophobic material) together with the one or more surfactants and one or more pharmaceutically active agents and/or one or more chemiluminescent disease-detecting systems. As one skilled in the art will readily appreciate, the bulk hydrophobic material possesses a specific melting point. However, the process is carried out such that the resulting hydrophobic particles will have a melting point below the melting point of the respective hydrophobic material. In general, the resulting hydrophobic particles will have a melting point of no more than about 81° C. In one embodiment, suitable hydrophobic material includes those having a melting point of from about 21° C. to about 33° C.

The resulting hydrophobic particles are solid hydrophobic particles having a mean particle size ranging from about 5 to about 50 nm. In one embodiment, the resulting hydrophobic particles will have a mean particle size ranging from about 7 to about 15 nm. In one embodiment, the resulting hydrophobic particles will have a mean particle size ranging from about 15 to about 25 nm. In one embodiment, the resulting hydrophobic particles will have a mean particle size ranging from about 25 to about 50 nm. It is also contemplated herein that the resulting hydrophobic particles can be of two or more distinct sizes to maximize skin penetration, i.e., to design a product that would target layers of differing depths of the skin, and also surface protection of the skin cells by the larger particles.

In general, the amount of hydrophobic material used to form the hydrophobic particles can range from about 1% to about 10% (wt./wt.). In one embodiment, the amount of hydrophobic material used to form the hydrophobic particles ranges from about 2.5 to about 5% (wt./wt.).

Hydrophilic Coating

The hydrophilic coating is derived from any amphiphilic material known in the art that is pharmaceutically acceptable surfactant. Suitable amphiphilic material include, by way of example, anionic, cationic, non-ionic and amphoteric surfactants. The term “surfactant” refers to any molecule having both a polar head group, which energetically prefers solvation by water, and a hydrophobic tail, which is not well solvated by water. For example, a “cationic surfactant” refers to a surfactant with a cationic head group, while an “anionic surfactant” refers to a surfactant with an anionic head group.

Suitable anionic surfactants include any anionic surfactant known in the art. Useful anionic surfactants include, but are not limited to, sulfates, sulfonates, phosphates, carboxylates and the like. Representative examples of anionic surfactants include alkyl or alkenyl ether sulfates, alkyl- or alkenylsulfates, sulfonate fluorosurfactants, alkylbenzenesulfonates, olefinsulfonates, alkanesulfonates, carboxylate fluorosurfactants, saturated or unsaturated fatty acid salts (soaps), alkyl or alkenyl ether carboxylates, α-sulfofatty acids, N-acylaminoacid surfactants, alkyl aryl ether phosphates, alkyl ether phosphates, phosphate mono- or diester surfactants, and sulfosuccinate ester surfactants. Examples of counter ions for the anionic residual groups in the above-described surfactants include alkali metal ions such as sodium ion and potassium ion; alkaline earth metal ions such as calcium ion and magnesium ion; ammonium ion; and alkanolamines having 1 to 3 alkanol groups each having 2 or 3 carbon atoms (for example, monoethanolamine, diethanolamine, triethanolamine, triisopropanolamine, and the like). Specific examples of such anionic surfactants include, dioctyl sodium sulfosuccinate; perfluorooctanesulfonate (PFOS), perfluorobutanesulfonate, sodium stearate, sodium lauroyl sarcosinate, perfluorononanoate, and perfluorooctanoate (PFOA or PFO).

Suitable cationic surfactants include any cationic surfactant known in the art. Useful cationic surfactants include, but are not limited to, pH-dependent primary, secondary or tertiary amines (primary amines become positively charged at pH<10, secondary amines become charged at pH<4) and quaternary ammonium salts represented by the following formula:

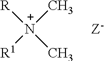

wherein Z⁻ is an anion, and R and R¹ are the same or different and is hydrogen, a substituted or unsubstituted alkyl group, a substituted or unsubstituted fluoroalkyl group, a substituted or unsubstituted cycloalkyl group, a substituted or unsubstituted aryl group, a substituted or unsubstituted heteroaryl group, a substituted or unsubstituted arylalkyl group, a substituted or unsubstituted heteroarylalkyl group, or —(CH₂)ₙ—R′, wherein R′ represents independently for each occurrence a substituted or unsubstituted cycloalkyl group, a substituted or unsubstituted aryl group, or a substituted or unsubstituted heteroaryl group and n represents independently for each occurrence an integer in the range 1 to 10 inclusive, with a proviso that at the same time, both R groups are not hydrogen atoms or benzyl groups or are not lower alkyl groups having 1 to 3 carbon atoms. In one embodiment, one of R and R¹ can be an alkyl group, especially a linear alkyl group having 12 to 24 carbon atoms, and the other one can be a lower alkyl group having 1 to 3 carbon atoms, especially a methyl group. Examples of the anion Z⁻include halide ions such as chloride ion and bromide ion; and organic anions such as ethyl sulfate ion and methyl carbonate ion.

Representative examples of cationic surfactants include, but are not limited to, cetyl trimethylammonium chloride, cetyl trimethylammonium bromide, stearyl trimethylammonium chloride, arachyl trimethylammonium chloride, behenyl trimethylamionium chloride, cetylpyridinium chloride, polyethoxylated tallow amine, benzalkonium chloride, benzothonium chloride, 5-Bromo-5-nitro-1,3-dioxane; dimethyldioctadecylammonium chloride, dioctadecyldimethylammonium bromide, octenidine dihydrochloride and the like.

Suitable non-ionic surfactants include any non-ionic surfactant known in the art. Useful non-ionic surfactants include, but are not limited to, fatty alcohols (e.g., cetyl alcohol, stearyl alcohol, cetostearyl alohol (consisting predominantly of cetyl and stearyl alcohols), and oleyl alcohol), polyoxyalkylene alkyl ethers (e.g. BRIJ 97® and CETOMACROGOL 1000®), polyoxyalkylene alkenyl ethers, polyoxyethylene glycol octylphenol ethers (e.g., Triton X-100), polyethylene glycol alkyl ethers (e.g., octaethylene glycol monododecyl ether, and pentaethylene glycol monododecyl ether), polyglyceryl fatty acid esters, higher fatty acid mono- or diethanolamides, polyoxyethylene hydrogenated castor oil, polyoxyethylene sorbitan fatty acid esters (e.g. TWEEN 20®), polyoxyethylene fatty acid esters (e.g. MYRJ 52®), sorbitan esters (e.g. SPAN 80®), sucrose esters (e.g. WASAG ESTER 7®), alkyl-saccharide-based surfactants, alkylamine oxides, and alkylamidoamine oxides.

Suitable amphoteric surfactants include any amphoteric surfactant known in the art. Useful amphoteric surfactants include, but are not limited to, imidazoline-based amphoteric surfactants, carbobetaine-based amphoteric surfactants, amidobetaine-based amphoteric surfactants, sulfobetaine-based amphoteric surfactants, hydroxysulfobetaine-based amphoteric surfactants, and amidosulfobetaine-based amphoteric surfactants. Representative examples of amphoteric surfactants include (3-[(3-Cholamidopropyl)dimethylammonio]-1-propanesulfonate) and cocamidopropyl hydroxysultaine.

In general, the amount of hydrophilic material used to form the hydrophilic coating can vary widely, e.g., an amount ranging from about 1% to about 10% by weight. In one embodiment, the amount of hydrophilic material used to form the hydrophilic coating ranges from about 2.5% to about 5% by weight.

Pharmaceutically Active Agents (Drugs)

The nanoemulsion can include one or more pharmaceutically active agents incorporated therein. In the event that the drugs are hydrophobic, then the hydrophobic drugs will be incorporated into the hydrophobic particle during the production process. If the drugs are hydrophilic, then the hydrophilic drugs will be attached to the hydrophilic coating. Suitable drugs for incorporating into the nanomulsion include, but are not limited to, those that improve or eradicate age spots, keratoses and wrinkles; local analgesics and anesthetics; antiacne agents; antibacterials; antiyeast agents; antifungal agents; antiviral agents; antidandruff agents; antidermatitis agents; antihistamine agents; antipruritic agents; antiemetics; antimotionsickness agents; anfiinflammatory agents; antihyperkeratolytic agents; antiperspirants; antipsoriatic agents; antiseborrheic agents; hair conditioners and hair treatment agents; antiaging and antiwrinkle agents; sunblock and sunscreen agents; skin lightening agents; depigmenting agents; vitamins; corticosteroids; tanning agents; humectants; hormones; retinoids; gum disease or oral care agents; topical cardiovascular agents; corn, callus and wart removing agents; and depilating agents. Particular drugs within the therapeutic categories can be found in standard pharmacology textbooks such as Remington: The Science and Practice of Pharmacy, (20th Ed. 2000).

Examples of the above agents include, but are not limited to, abacavir, acebutolol, acetaminophen, acetaminosalol, acetazolamide, acetohydroxamic acid, acetylsalicylic acid, acitretin, aclovate, acrivastine, actiq, acyclovir, adapalene, adefovir dipivoxil, adenosine, albuterol, alfuzosin, allopurinol, alloxanthine, almotriptan, alprazolam, alprenolol, aluminum acetate, aluminum chloride, aluminum chlorohydroxide, aluminum hydroxide, amantadine, amiloride, aminacrine, aminobenzoic acid (PABA), aminocaproic acid, aminosalicylic acid, amiodarone, amitriptyline, amlodipine, amocarzine, amodiaquin, amorolfine, amoxapine, amphetamine, ampicillin, anagrelide, anastrozole, anthralin, apomorphine, aprepitant, arbutin, aripiprazole, ascorbic acid, ascorbyl palmitate, atazanavir, atenolol, atomoxetine, atropine, azathioprine, azelaic acid, azelastine, azithiromycin, bacitracin, beclomethasone dipropionate, bemegride, benazepril, bendroflumethiazide, benzocaine, benzonatate, benzophenone, benztropine, bepridil, betamethasone dipropionate, betamethasone valerate, brimonidine, brompheniramine, bupivacaine, buprenorphine, bupropion, burimamide, butenafine, butoconazole, cabergoline, caffeic acid, caffeine, calcipotriene, camphor, candesartan cilexetil, capsaicin, carbamazepine, cefditoren pivoxil, cefepime, cefpodoxime proxetil, celecoxib, cetirizine, cevimeline, chitosan, chlordiazepoxide, chlorhexidine, chloroquine, chlorothiazide, chloroxylenol, chlorpheniramine, chlorpromazine, chlorpropamide, ciclopirox, cilostazol, cimetidine, cinacalcet, ciprofloxacin, citalopram, citric acid, cladribine, clarithromycin, clemastine, clindamycin, clioquinol, clobetasol propionate, clomiphene, clonidine, clopidogrel, clotrimazole, clozapine, cocaine, codeine, cromolyn, crotamiton, cyclizine, cyclobenzaprine, cycloserine, cytarabine, dacarbazine, dalfopristin, dapsone, daptomycin, daunorubicin, deferoxamine, dehydroepiandrosterone, delavirdine, desipramine, desloratadine, desmopressin, desoximetasone, dexamethasone, dexmedetomidine, dexmethylphenidate, dexrazoxane, dextroamphetamine, diazepam, dicyclomine, didanosine, dihydrocodeine, dihydromorphine, diltiazem, 6,8-dimercaptooctanoic acid (dihydrolipoic acid), diphenhydramine, diphenoxylate, dipyridamole, disopyramide, dobutamine, dofetilide, dolasetron, donepezil, dopa esters, dopamide, dopamine, dorzolamide, doxepin, doxorubicin, doxycycline, doxylamine, doxypin, duloxetine, dyclonine, econazole, eflornithine, eletriptan, emtricitabine, enalapril, ephedrine, epinephrine, epinine, epirubicin, eptifibatide, ergotarnine, erythromycin, escitalopram, esmolol, esomeprazole, estazolam, estradiol, ethacrynic acid, ethinyl estradiol, etidocaine, etomidate, famciclovir, famotidine, felodipine, fentanyl, ferulic acid, fexofenadine, flecamide, fluconazole, flucytosiine, fluocinolone acetonide, fluocinonide, 5-fluorouracil, fluoxetine, fluphenazine, flurazepam, fluvoxamine, formoterol, furosemide, galactarolactone, galactonic acid, galactonolactone, galantamine, gatifloxacin, gefitinib, gemcitabine, gemifloxacin, glycolic acid, griseofulvin, guaifenesin, guanethidine, N-guanylhistamine, haloperidol, haloprogin, hexylresorcinol, homatropine, homosalate, hydralazine, hydrochlorothiazide, hydrocortisone, hydrocortisone 21-acetate, hydrocortisone 17-butyrate, hydrocortisone 17-valerate, hydromorphone, hydroquinone, hydroquinone monoether, hydroxyzine, hyoscyamine, hypoxanthine, ibuprofen, ichthammol, idarubicin, imatinib, imipramine, imiquimod, indinavir, indomethacin, irbesartan, irinotecan, isoetharine, isoproterenol, itraconazole, kanamycin, ketamine, ketanserin, ketoconazole, ketoprofen, ketotifen, kojic acid, labetalol, lactic acid, lactobionic acid, lamivudine, lamotrigine, lansoprazole, letrozole, leuprolide, levalbuterol, levofloxacin, lidocaine, linezolid, lobeline, loperamide, losartan, loxapine, lysergic diethylamide, mafenide, malic acid, maltobionic acid, mandelic acid, maprotiline, mebendazole, mecamylamine, meclizine, meclocycline, memantine, menthol, meperidine, mepivacaine, mercaptopurine, mescaline, metanephrine, metaproterenol, metaraminol, metformin, methadone, methamphetamine, methotrexate, methoxamine, methyldopa esters, methyldopamide, 3,4-methylenedioxymethamphetamine, methyllactic acid, methyl nicotinate, methylphenidate, methyl salicylate, metiamide, metolazone, metoprolol, metronidazole, mexiletine, miconazole, midazolam, midodrine, miglustat, minocycline, minoxidil, mirtazapine, mitoxantrone, moexiprilat, molindone, monobenzone, morphine, moxifloxacin, moxonidine, mupirocin, nadolol, naftifine, nalbuphine, nalmefene, naloxone, naproxen, nefazodone, nelfinavir, neomycin, nevirapine, nicardipine, nicotine, nifedipine, nimodipine, nisoldipine, nizatidine, norepinephrine, nystatin, octopamine, octreotide, octyl methoxycinnamate, octyl salicylate, ofloxacin, olanzapine, olmesartan medoxomil, olopatadine, omeprazole, ondansetron, oxiconazole, oxotremorine, oxybenzone, oxybutynin, oxycodone, oxymetazoline, padimate O, palonosetron, pantothenic acid, pantoyl lactone, paroxetine, pemoline, penciclovir, penicillamine, penicillins, pentazocine, pentobarbital, pentostatin, pentoxifylline, pergolide, perindopril, permethrin, phencyclidine, phenelzine, pheniramine, phenmetrazine, phenobarbital, phenol, phenoxybenzamine, phentolamine, phenylephrine, phenylpropanolarnine, phenyloin, physostigmine, pilocarpine, pimozide, pindolol, pioglitazone, pipamazine, piperonyl butoxide, pirenzepine, podofilox, podophyllin, pratipexole, pramoxine, prazosin, prednisone, prenalterol, prilocalne, procainamide, procaine, procarbazine, promazine, promethazine, promethazine propionate, propafenone, propoxyphene, propranolol, propylthiouracil, protriptyline, pseudoephedrine, pyrethrin, pyrilamine, pyrimethamine, quetiapine, quinapril, quinethazone, quinidine, quinupristin, rabeprazole, reserpine, resorcinol, retinal, 13-cis retinoic acid, retinoic acid, retinol, retinyl acetate, retinyl palmitate, ribavirin, ribonic acid, ribonolactone, rifampin, rifapentine, rifaximin, riluzole, rimantadine, risedronic acid, risperidone, ritodrine, rivastigmine, rizatriptan, ropinirole, ropivacaine, salicylamide, salicylic acid, salmeterol, scopolamine, selegiline, selenium sulfide, serotonin, sertindole, sertraline, sibutramine, sildenafil, sotalol, streptomycin, strychnine, sulconazole, sulfabenz, sulfabenzamide, sulfabromomethazine, sulfacetamide, sulfachlorpyridazine, sulfacytine, sulfadiazine, sulfadimethoxine, sulfadoxine, sulfaguanole, sulfalene, sulfamethizole, sulfamethoxazole, sulfanilamide, sulfapyrazine, sulfapyridine, sulfasalazine, sulfasomizole, sulfathiazole, sulfisoxazole, tadalafil, tamsulosin, tartaric acid, tazarotene, tegaserol, telithromycin, telmisartan, temozolomide, tenofovir disoproxil, terazosin, terbinafine, terbutaline, terconazole, terfenadine, tetracaine, tetracycline, tetrahydrozoline, theobromine, theophylline, thiabendazole, thioridazine, thiothixene, thymol, tiagabine, timolol, timidazole, tioconazole, tirofiban, tizanidine, tobramycin, tocamide, tolazoline, tolbutamide, tolnaftate, tolterodine, tramadol, tranylcypromine, trazodone, triamcinolone acetonide, triamcinolone diacetate, triamcinolone hexacetonide, triamterene, triazolam, triclosan, triflupromazine, trimethoprim, trimipramine, tripelennamine, triprolidine, tromethamine, tropic acid, tyramine, undecylenic acid, urea, urocanic acid, ursodiol, vardenafil, venlafaxine, verapamil, vitamin E acetate, voriconazole, warfarin, xanthine, zafirlukast, zaleplon, zinc pyrithione, ziprasidone, zolmitriptan and zolpidem.

The concentration of the one or more pharmaceutically active agents in the topical composition is a concentration sufficient to provide the desired cosmetic, dermatological or disease treating effect, which may vary depending on the desired cosmetic condition, dermatological disorder or disease being treated, the size of the patient, and other factors. In general, the concentration of the one or more pharmaceutically active agent can range from about 0.01% to about 20% by weight, based on the total weight of the oil phase. In one embodiment, the concentration of the one or more pharmaceutically active agent can range from about 1 to about 10% by weight, based on the total weight of the oil phase (ie. wax or butter).

Chemiluminescent Disease-Detecting Systems

The nanoemulsion can also include one or more chemiluminescent disease-detecting systems incorporated therein for detecting a disease or condition in a host. For example, hydrogen peroxide (H₂O₂) is a reactive oxygen metabolic by-product that can serve as a key regulator for a number of oxidative stress related states. Hydrogen peroxide is believed to be over-produced by cells at the early stages of most diseases such as asthma, inflammatory arthritis, atherosclerosis, diabetic vasculopathy, osteoporosis, and a number of neurodegenerative diseases. In addition, overproduction of hydrogen peroxide can occur in the development of damage caused to skin by exposure to ultraviolet radiation. Thus, detecting low levels of hydrogen peroxide in the skin could serve as an early warning indicator for skin cancer. The topical composition of the present invention can therefore be used as a simple, all-purpose diagnostic tool for detecting diseases.

Accordingly, in one embodiment, the one or more chemiluminescent disease-detecting systems include a peroxide-reactive compound and a light-emitting material. By incorporating a peroxide-reactive compound and a light-emitting material into the hydrophobic particle, a three-component reaction in vivo can advantageously be achieved. When the hydrophobic particles bump into hydrogen peroxide, they will excite the light-emitting material, e.g., dye, which then emits photons that exhibits chemiluminescence in the presence of the peroxide. Thus, if a doctor would see a significant amount of chemiluminescence in the area in which the topical composition was applied, the doctor would know that the patient may be presenting early signs of a disease in that area of the body.

In one embodiment, a suitable peroxide-reactive compound is an oxalate ester. Useful oxalate esters include compounds represented by the formula:

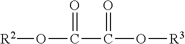

wherein R² and R³ are independently a substituted or unsubstituted aryl group, or a substituted or unsubstituted heteroaryl. The aryl or heteroaryl group may be substituted with, for example, hydrogen, hydroxy, halide (e.g., chloride, bromide, fluoride, etc.), a carbonyl group, an optionally substituted amine, optionally substituted alkyl, optionally substituted alkoxy, cyano, and/or nitro group. Representative examples of heteroaryl groups for use herein include, by way of example, a substituted or unsubstituted stable 3 to about 30 membered ring radical, containing carbon atoms and from one to five heteroatoms, e.g., nitrogen, phosphorus, oxygen, sulfur and mixtures thereof. Suitable heteroaryl groups may be a monocyclic, bicyclic or tricyclic ring system, which may include fused, bridged or spiro ring systems, and the nitrogen, phosphorus, carbon, oxygen or sulfur atoms in the heterocyclic ring radical may be optionally oxidized to various oxidation states.

Representative examples of oxalate esters include, but are not limited to, iptycene compounds, bis(2-carbopentyloxy-3,5,6-trichlorophenyl oxalate, bis(2-nitrophenyl) oxalate, bis(2,4-dinitrophenyl) oxalate, bis(2,6-dichloro-4-nitrophenyl) oxalate, bis(2,4,6-trichlorophenyl) oxalate, bis(3-trifluoromethyl-4-nitrophenyl) oxalate, bis(2-methyl-4,6-dinitrophenyl) oxalate, bis(1,2-dimethyl-4,6-dinitrophenyl) oxalate, bis(2,4-dichlorophenyl) oxalate, bis(2,5-dinitrophenyl) oxalate, bis(2-formyl-4-nitrophenyl) oxalate, bis(pentachlorophenyl) oxalate, bis(pentafluorophenyl) oxalate, bis(1,2-dihydro-2-oxo-1-pyridyl) glyoxal, bis-N-phthalmidyl oxalate, bis(6-carbopentoxy-2,4,5-trichlorophenyl)oxalate (CPPO), bis(2,4,5-trichloro-6-carbobutoxyphenyl) oxalate, bis(2,4,6-trichlorophenyl)oxalate (TCPO), bis(2,4,5-trichloro-6-carbopentoxyphenyl) oxalate, bis(2,4,5-trichloro-6-carbobutoxyphenyl) oxalate, bis(2,4,6-trichlorophenyl) oxalate, 1,1-oxalyldiimidazole and phthalimido 3,6,6-trisulfo-2-naphthyl oxalate.

Other examples of peroxide-reactive compounds include 5-amino-2,3-dihydrophthalazine-1,4-dione or 3-aminophthalhydrazide (luminol), Cyalume® (containing diphenylethandioate, a dye, and other components) 2,4,5-triphenyll midazole (lophine), 10,10′-dialkyl-9,9′-biacridinium salts (lucigenin), and 9-chlorocarbonyl-10-methylacridinium chloride (rosigenin), and the like. In one embodiments, the peroxide-reactive compound is bis(2,4,6-trichlorophenyl) oxalate, bis(2-carbopentyloxy-3,5,6-trichlorophenyl)oxalate, oxalic acid, bis[2,4,5-trichloro-6-(pentyloxycarbonyl)phenyl]ester and bis(pentafluorophenyl) oxalate. In one embodiment, bis(2,4,6-trichlorophenyl) oxalate is the peroxide-reactive compound. In another embodiment, bis(2-carbopentyloxy-3,5,6-trichlorophenyl) oxalate is the peroxide-reactive compound.

In general, the peroxide-reactive compound is incorporated into the nanoemulsion in an amount ranging from about 1% to about 20% by weight of the hydrophobic oil phase. In one embodiment, the peroxide-reactive compound is incorporated into the nanoemulsion in an amount ranging from about 10% to about 20% by weight of the oil phase.

Suitable light-emitting materials may be any luminescent material, including dyes, oligomers, polymers, and the like combinations thereof. The light-emitting material may be selected to exhibit certain properties, such as a particular emission wavelength, high quantum yield, high output light efficiency when formulated in a peroxide reactive system, and/or compatibility (e.g., solubility) with one or more components of the nanoemulsion. In one embodiment, the light-emitting material may be selected to exhibit a high quantum yield, for example, when present in a nanoemulsion having a high concentration of light-emitting material. As used herein, the “quantum yield” of a material refers to the total emission produced by the material, i.e., the number of photons emitted per absorbed photon. In some embodiments, the light emitting material may be selected to exhibit a high output light efficiency when formulated in a peroxide reactive system. As used herein, “output light efficiency” of a material in the nanoemulsion refers to the yield of output light (e.g., observable light) produced by the nanoemulsion in the presence of a peroxide, i.e., the efficiency of the interaction between the peroxide and the system in generating light.

In some cases, light-emitting materials may be any compound which has a determinable emission of light (e.g., chemiluminescence, fluorescence, phosphorescence), with an emission spectrum between, for example, about 400 to about 800 nm. In one embodiment, the light-emitting material is a fluorescent dye. In another embodiment, suitable light-emitting materials include, but are not limited to, anthracene, benzanthracene, phenanthrene, naphthacene, pentacene, diphenylanthracene, 9,10-bis(phenylethynyl)anthracene, substituted derivatives thereof, and the like. Examples of substituents include phenyl, lower alkyl, halide, cyano, alkoxy, and other substituents which do not interfere with the light-emitting reaction described herein.

In one embodiment, the light-emitting material may be a conjugated polymer, such as poly(phenylene-ethynylene), poly(phenylene-vinylene), poly(p-phenylene), polythiophene, other poly(arylene)s, substitute derivatives thereof, and the like. The light-emitting capability of such polymers are known in the art, and can be selected to suit a particular application.

In one embodiment, the light-emitting material may be covalently bound to the peroxide-reactive material. In some embodiments, the light-emitting material may be covalently bound to the hydrophobic particle.

In general, the light-emitting material is incorporated into the nanoemulsion in an amount ranging from about 0.01 to about 10 mg/ml based on the volume of the hydrophobic material. In one embodiment, the light-emitting material is incorporated into the nanoemulsion in an amount ranging from about 0.1 to about 1 mg/ml based on the volume of the hydrophobic material.

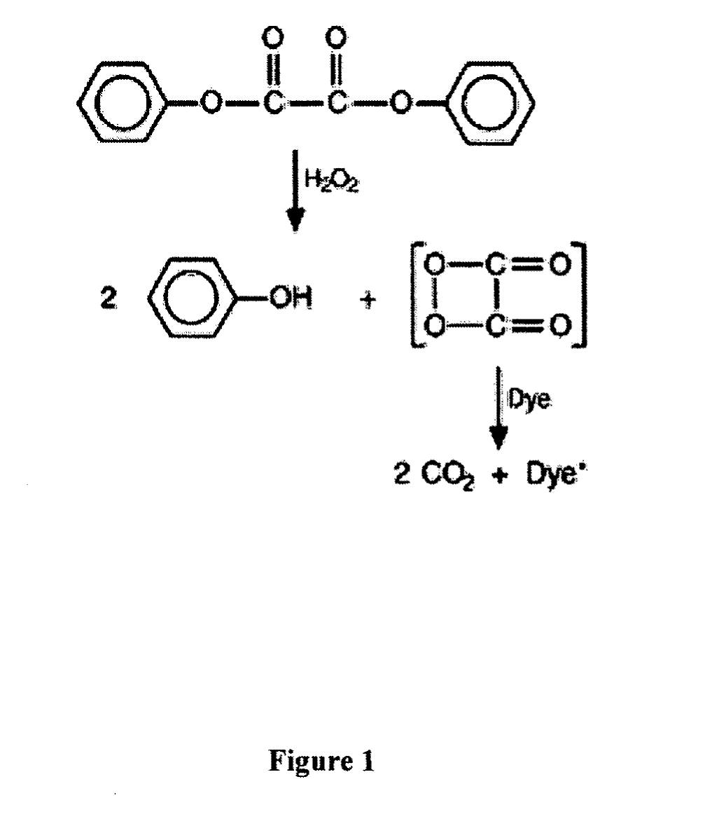

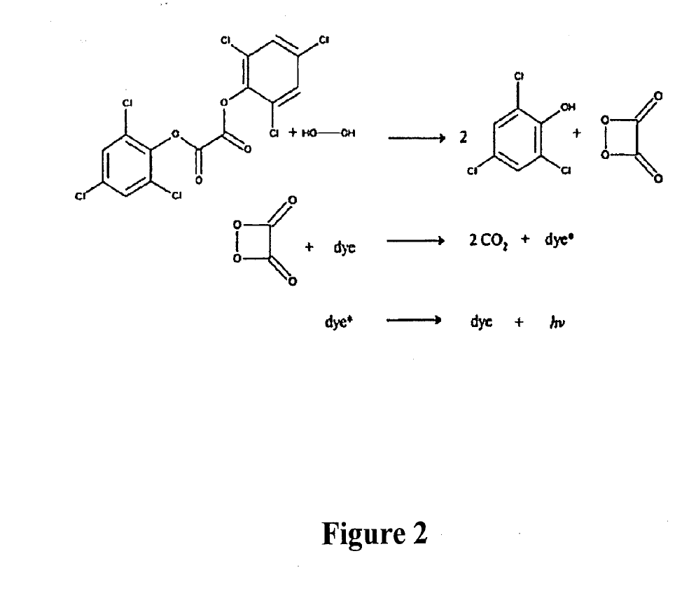

FIGS. 1 and 2 show a chemical reaction that produces chemiluminescence. In general, the mechanism is that first the phenyl oxalate ester and hydrogen peroxide (H₂O₂) react to form a peroxy acid ester and phenol; and then the peroxy acid ester decomposes to form more phenol and a highly energetic intermediate, presumed to be a cyclic compound containing a four-membered ring dimer of CO₂. As the cyclic dimer decomposes into two CO₂ molecules, it gives up its energy to a waiting dye molecule, which then fluoresces.

As shown in FIG. 2, the chemical reaction taking place includes a solution of a phenyl oxalate ester (commonly bis(2,4,5-trichlorophenyl-6-carbopentoxyphenyl)oxalate (CPPO), a fluorescent dye that determines the color of light, and hydrogen peroxide (H₂O₂). The hydrogen peroxide reacts with the phenyl oxalate ester producing carbon dioxide, a phenol and, most importantly, releasing energy. This energy is absorbed by the dye, exciting electrons in the dye's molecules to a higher energy level. Once at the higher energy level, the electrons immediately lose the energy they absorbed and fall to lower energy levels. As the electrons fall back to lower energy levels, the energy that is lost is transformed into electromagnetic radiation, some of which is visible light.

The dye must gain energy from the breakdown of the CPPO. For light to be seen in the visible spectrum, the radiation emitted must be between 400 nm and 700 nm. This range of wavelengths has a corresponding range of energy required between 170 kJ/mol and 300 kJ/mol. This energy is released from a high energy intermediate that forms during the reaction and is transferred to the dye. The intermediate that forms when the CPPO is oxidized by hydrogen peroxide is called 1,2-dioxetane-3,4-dione. This compound is a four membered ring structure. As in most four membered rings, the compound is considered high in energy because of the strain on the bonds of the molecule forcing them into the ring. Although the compounds are different, they all share the common trait of being highly conjugated systems. This conjugation allows electrons to move easily because of the small gap between the highest occupied molecular orbital (HOMO) and the lowest unoccupied molecular orbital (LUMO). The energy produced as the four-membered ring structure decomposes into two CO₂ molecules is enough to move the electrons across the gap.

Since the color of the light depends on the fluorescer selected, peroxyoxalate chemiluminescence can be formulated in many desired colors. For example, a blue color can be produced using 9,10 diphenylanthracene, green color can be produced using 9,10-bis(phenylethynyl)anthracene, and yellow can be produced using either of 1-chloro-9,10-bis(phenylethynyl)anthracene or Rubrene [5,6,11,12-tetraphenylnaphthacene], orange can be produced using 5,12-bis(phenylethynyl)-napthacene.

In another embodiment, the chemiluminescent disease-detecting systems can detect a number of other biomarkers when combined with an appropriate source of energy capable of converting an enzyme that catalyzes an oxidation-reduction reaction to obtain hydrogen peroxide. In one preferred embodiment, a suitable source is an oxidase. An oxidase is any enzyme that catalyzes an oxidation-reduction reaction involving molecular oxygen (O₂) as the electron acceptor. In these reactions, oxygen is reduced to hydrogen peroxide (H₂O₂). For example, glucose oxidase (GOx) is an enzyme that catalyzes the oxidation of β-D-glucose to D-glucono-1,5-lactone with O₂ as the electron and proton acceptor, creating hydrogen peroxide (H₂O₂) as a byproduct. In such embodiment, a concentration of glucose in the presence of the GOx is established.

Suitable sources of energy for use herein include, but are not limited to, one or more of malate oxidase, hexose oxidase, glucose oxidase, glutamate oxidase, cholesterol oxidase, aryl-alcohol oxidase, L-gulonolactone oxidase, galactose oxidase, pyranose oxidase, L-sorbose oxidase, pyridoxine 4-oxidase, alcohol oxidase, catechol oxidase, (S)-2-hydroxy-acid oxidase, ecdysone oxidase, choline oxidase, secondary-alcohol oxidase, 4-hydroxymandelate oxidase, long-chain-alcohol oxidase, glycerol-3-phosphate oxidase, thiamine oxidase, hydroxyphytanate oxidase, nucleoside oxidase, N-acylhexosamine oxidase, polyvinyl-alcohol oxidase, D-arabinono-1,4-lactone oxidase, vanillyl-alcohol oxidase, nucleoside oxidase, D-mannitol oxidase, xylitol oxidase, monoamine oxidase, xanthine oxidase such as hypoxanthine, L-gulonolactone oxidase, and lysyl oxidase and the like and mixtures thereof.

Process for Preparing the Nanoemulsion

The nanoemulsions formed herein are an emulsified system which undergoes a phase transition from a water-in-oil (W/O) emulsion to an oil-in-water (O/W) emulsion. At the phase inversion temperature, the interfacial tension between water and oil reaches a minimum. If the system is agitated during this minimum, the continuous oil phase breaks up into nanoscopic droplets. Thus, to obtain the nanoemulsion described herein, an oil/water/surfactant system containing the foregoing components, i.e., hydrophobic material, hydrophilic material, drug and/or chemiluminescent disease-detecting system and optional penetration enhancing compounds is heated above the phase inversion temperature, e.g., a temperature ranging from about 30° C. to about 90° C., and then stirred while it cools back to room temperature. To obtain the solid hydrophobic particles with a hydrophilic coating, the melting point of the oil phase is kept below the phase inversion temperature, e.g., a temperature ranging from about 15° C. to about 80° C. Methods for obtaining such a nanoemulsion are described, for example, in Forgiarini et al., “Studies of the relation between phase behavior and emulsification methods with nanoemulsion formation”, Colloidal Polym. Sci. Vol 115, pp. 36-39 (2000); Tadros et al., “Formation and stability of nano-emulsions”, Elsevier, Advances in Colloidal And Interface Science, pp. 303-318 (2004), and Forgiarini et al., “Formation of Nano-emulsions by Low-Energy Emulsification Methods at Constant Temperature”, Langmuir Vol. 17, pp. 2076-2083 (2001), the contents of which are incorporated by reference herein.

Topical Compositions

The topical compositions of the present invention can further contain one or more pharmaceutically acceptable excipients or carriers. Suitable pharmaceutically acceptable excipients or carriers include thickening agents, pigments, preservatives, pH adjusting agents, hydrating agents, ultraviolet-absorbing agents, and the like.

A thickening agent can be, for example, a polymer that is water soluble or that generates a colloidal solution in water. Useful thickening agents include, but are not limited to, polymers or copolymers unsaturated carboxylic acids or unsaturated esters, polysaccharide derivatives, gums, colloidal silicates, polyethylene glycols (PEG) and their derivatives, polyvinylpyrrolidones and their derivatives, polyacrylamides and their derivatives, polyacrylonitriles, hydrophilic silica gels, or mixtures thereof. Examples of thickening agents include acrylic and/or methacrylic polymers or copolymers, vinylcarboxylic polymers, polyglyceryl acrylates or methacrylates, polyacrylamides derivatives, cellulose or starch derivatives, chitin derivatives, alginates, hyaluronic acid and its salts, chonodroitin sulphates, xanthan, gellan, Rhamsan, karaya or guar gum, carob flour, and colloidal aluminum magnesium silicates of the montmorillonite type.

In one embodiment, a thickening agent includes vinylcarboxylic polymers sold under the tradename CARBOPOL® (Goodrich), acrylic acid/ethyl acrylate copolymers, acrylic acid/stearyl methacrylate copolymers, carboxymethylcellulose, hydroxymethylcellulose, hydroxypropylcellulose, microcrystalline cellulose, hydroxypropyl guar, colloidal hectorites, bentonites, and the like.

Suitable pigments include, but are not limited to, inorganic pigments, organic pigments, or nacreous pigments. Examples of inorganic pigments include titanium dioxide, black, yellow, red or brown iron oxide, manganese violet, ultramarine violet, ultramarine blue, chromium oxide, and the like. Examples of organic pigments include D & C Red No. 3, No. 6, No. 7, No. 9, No. 13, No. 19, No. 21, No. 27, No. 30, or No. 36, or alternatively carbon black. Examples of nacreous pigments include white nacreous pigments, such as mica coated with titanium oxide or with bismuth oxychloride. Colored nacreous pigments, such as titanium mica colored with iron oxides or with chromium oxide, titanium mica colored with an organic pigment of the above-mentioned type, or alternatively, nacreous pigments based on bismuth oxychloride, also can be used.

A preservative can be used to prevent, for example, bacterial attack. Suitable preservatives include, but are not limited to, butylparaben, propylparaben, chlorocresol, sorbic acid, benzoic acid and the like and mixtures thereof.

Suitable basic pH adjusters include, but are not limited to, ammonia; mono-, di-, and tri-alkyl amines; mono-, di-, and tri-alkanolamines; alkali metal and alkaline earth metal hydroxides; and mixtures thereof. Examples of basic pH adjusters include ammonia; sodium, potassium, and lithium hydroxide; monoethanolamine; triethylamine; isopropanolamine; diethanolamine; and triethanolamine.

Suitable acidic pH adjusters include, but are not limited to, mineral acids, organic carboxylic acids and the like. Examples of mineral acids include citric acid, hydrochloric acid, nitric acid, phosphoric acid, and sulfuric acid.

In one embodiment, the topical compositions of the present invention can be formulated in a semi-liquid formulation. Examples of such topical compositions include, but are not limited to, a topical solution, spray, mist, drops and the like. Accordingly, the topical compositions can be administered by way of injection by needle into a certain area of the body in the case where it is a topical solution. If the nanoemulsion encounters hydrogen peroxide, the hydrophobic particles would emit light.

In one embodiment, the topical compositions of the present invention can be in a semisolid form such as, for example, gels, creams, lotions, suspensions, emulsions, ointments, foams, pastes and the like. Thus, the topical compositions can be administered by way of a transdermal patch or can be applied to directly to area of the body in need of treatment.

Ointments, as is well known in the art of pharmaceutical formulation, are semi-solid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable-bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Preferred water-soluble ointment bases are prepared from polyethylene glycols of varying molecular weight; again, see Remington: The Science and Practice of Pharmacy for further information.

Creams, as also well known in the art, are viscous liquids or semi-solid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

As will be readily be understood by those skilled in the field of pharmaceutical formulation, gels are semi-solid, suspension-type systems. Gel forming agent for use herein can be any gelling agent typically used in the pharmaceutical art for topical semi solid dosage forms. Single-phase gels contain organic macromolecules distributed substantially uniformly throughout the carrier liquid, which is typically aqueous, but also can contain an alcohol and optionally an oil. In order to prepare a uniform gel, dispersing agents such as alcohol or glycerin can be added, or the gelling agent can be dispersed by tritration, mechanical mixing or stirring, or combinations thereof. The amount of gelling agents varies widely and will ordinarily range from about 0.1% to about 2.0% by weight, based on the total weight of the composition. The gel forming agent also works by the principle of copolymerization. Under alkaline pH, carbomer in presence of water undergoes cross linking and forms a gel like structure. The degree of polymerization is dependent upon the pH. At a threshold pH, the viscosities achieved by the polymer grade is the maximum.

Lotions, are preparations to be applied to the skin surface without friction, and are typically semi-liquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Pastes are semi-solid dosage forms in which the active agent is suspended in a suitable base. Depending on the nature of the base, pastes are divided between fatty pastes or those made from a single-phase aqueous gels. The base in a fatty paste is generally petrolatum or hydrophilic petrolatum or the like. The pastes made from single-phase aqueous gels generally incorporate carboxymethylcellulose or the like as a base.

In one embodiment, the topical compositions of the present invention may be used to treat a disease condition by applying a therapeutically effective amount of the topical composition to the area in need of treatment; wherein the nanoemulsion includes one or more pharmaceutically active agents. By applying the topical composition to the area of the host in need of treatment, the solid hydrophilic particles will melt and be absorbed into the skin The hydrophilic particles are of a size small enough to diffuse more deeply into the skin than larger particles. In this manner, the one or more pharmaceutically active agents can be released from the nanoemulsion and treat the disease condition.

The topical compositions of the present invention may be used to treat a wide variety of disease conditions or disorders as long as the disease or disorder is capable of being treated with a topical dermatological agent or at least suspected of being treatable with a topical dermatological agent. As noted above, in certain embodiments of the subject invention, a dermatological agent is not employed. The topical compositions of the present invention are suited to treat disease conditions that affect the skin and thus may be characterized as skin conditions or dermatological conditions. As used herein, the term “skin condition” and analogous terms are used broadly to refer to any condition of the skin in need of treatment, including those that do not involve inflammation of the skin, e.g., vitiligo. These skin disorders may be the result of a bacterial infection, viral infection, fungal infection, auto-immune response, allergenic response, idiopathic, and the like and may be acute or chronic lasting from a few minutes to a lifetime.

Accordingly, the topical compositions of the present invention can be used to treat any disease condition including those that originate at or are localized to the skin, as well as disease conditions that originate elsewhere in the body and/or are not localized to the skin In other words, disease conditions that may affect the skin may originate in another organ or system of the body, but may have manifestations or symptoms that affect the skin. Disease conditions that may be treated in accordance with one method of the present invention include, but are not limited to, psoriasis, eczema, vitiligo, atopic dermatitis, contact dermatitis, nummular dermatitis, generalized exfoliative dermatitis, stasis dermatitis, perioral dermatitis, morphoca, palmoplantar pustulosis, ichthyosis, keratoderma, warts, porokeratosis, Hailey-Hailey disease, pityriasis rubra pilaris, acanthosis nigricans, rosacea, lupus erythmatosus, Lichen simplex chronicus (neurodermatitis), alopecia, seborrheia, actinic keratosis, corns, pruritis, sun burn, urticaria, localized scratch dermatitis, staphylococcal disorders, ertsipelas, folliculitis, carbuncles, furuncles, erythrasma, candidiasis, dermatophyte infections, scabies, pediculosis, hypertrichosis, lichen planus, etc.

In another embodiment, the topical compositions of the present invention can be used to detect a disease or a condition in need of treatment. In general, an effective amount of a topical composition is contacted with a host; wherein the topical composition comprises a nanoemulsion of a plurality of hydrophobic particles having a hydrophilic coating therein, wherein the hydrophobic particles are derived from the same or different hydrophobic material and has a melting point below the melting point of the respective hydrophobic material, and further wherein the nanoemulsion further includes one or more chemiluminescent disease-detecting systems. As discussed above, the one or more chemiluminescent disease-detecting systems can be used to detect, for example, diseases such as asthma, inflammatory arthritis, atherosclerosis, diabetic vasculopathy, osteoporosis, a number of neurodegenerative diseases, skin cancer and the like.

It is further within the scope of the invention to provide a kit containing the apparatus and/or reagents necessary to carry out the test method of detecting a disease or a condition in need of treatment in the field. A complete kit would contain all of the equipment and consumables for conducting at least one test procedure. Thus, such a kit would include at a minimum a source of a topical composition comprising a nanoemulsion of a plurality of hydrophobic particles having a hydrophilic coating therein, wherein the hydrophobic particles are derived from the same or different hydrophobic material and has a melting point below the melting point of the respective hydrophobic material, and further wherein the nanoemulsion further includes one or more chemiluminescent disease-detecting systems; and (b) a light emitting detector for detecting a disease or a condition in the host. In general, light emitting detectors include CCD cameras, photodiodes, UV-vis spectrometers, and hyperspectral cameras.

The following examples are provided to enable one skilled in the art to practice the invention and are merely illustrative of the invention. The examples should not be read as limiting the scope of the invention as defined in the claims.

### Example 1

### Preparation of a Nanoemulsion

Into a 4 ml vial was dissolved a fluorescent dye, 9,10-bis(phenylethynyl)-antracene (1 mg), the active chemiluminescent material, 1-1′-oxalyldiimidazole (940 mg), methyl alcohol (950 ul), and decyl alcohol (50 ul). Next, the surfactant, Brij 97 (110 mg) and the wax, eicosane (100 mg), was added to the mixture. The mixture was heated to 60° C., above the melting point of eicosane and mixed until uniform. Distilled water (1.7 g) was added to the oily mixture. The vial was heated again to 60° C. and then the mixture was stirred continuously and allowed to cool to room temperature such that a phase inversion temperature was seen by noting the transition between a cloudy suspension at high temperatures, and a transparent nanoemulsion at room temperature. The particle size of the hydrophobic particles was 8 nm as determined by dynamic light scattering.

### Example 2

### Toxcity Testing

The nanoemulsion of Example 1 was then tested for toxicity. Human keratinocytes (HaCaT, Cell Lines Service) were cultured in Dulbeco's modified Eagle's medium with glucose and L-glutamine, without sodium pyruvate (Mediatech), 10% fetal bovine serum, 100 U/ml penicillin, and 100 μg/ml streptomycin. Cells were maintained in a humidified atmosphere at 37° C., 5% CO₂ and passaged at 80% confluence. HaCaT cells (MTT assay) were plated at a density of 4×10⁴ cells/well and allowed to equilibrate at 37° C. overnight. Next, 5 μl/well of the nanoemulsion of Example 1 was added to each replicate well. After 18 hours of exposure to the nanoemulsion preparation, cellular health was determined using the MTT assay, according to the manufacturer's instructions (ATCC). Briefly, MTT assay was added to the wells of the microplate and after two hours of incubation at 37° C., intracellular formazin crystals were solubilized with the manufacturer's detergent solution (ATCC). Absorbance values were obtained using the Safire2 microplate reader (Tecan US, Raleigh, N.C.) with a measurement wavelength of 570 nm and a reference wavelength of 700 nm, read from the bottom.

### Example 3

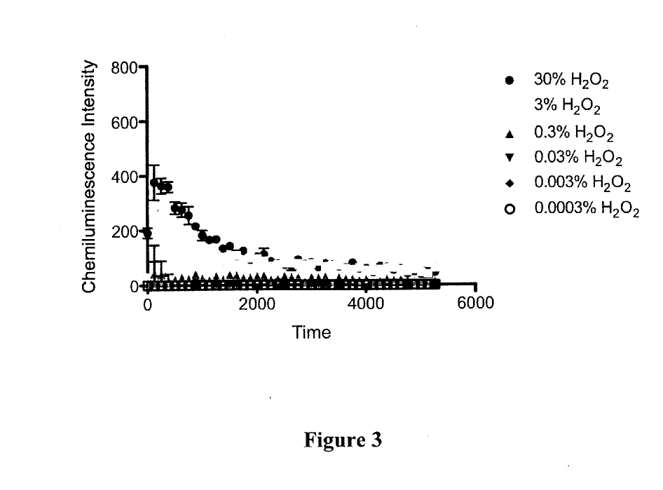

Chemiluminescence from exogenously added hydrogen peroxide was measured using the Tecan Safire2 microplate reader. The nanoemulsion (100 μl) of Example 1 was pipetted into six wells of a white, flat-bottom, 96-well microtiter plate. Various amount of hydrogen peroxide (H₂O₂) (i.e., 0.0003, 0.003% H₂O₂, 0.03% H₂O₂, 0.3% H₂O₂, 3% H₂O₂ and 30% H₂O₂) was diluted in distilled water and 5 μl of the diluted solution was added to each appropriate well. Sample chemiluminescence was immediately read using the Safire2 microplate reader (Tecan US, Raleigh, N.C.) and was measured every two minutes for sixty minutes. Values for duplicate wells were averaged and the standard deviation determined. FIG. 3 shows the results of the measurements.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

What is claimed is:

1. A topical composition comprising a nanoemulsion of a plurality of hydrophobic particles having a hydrophilic coating therein, wherein the hydrophobic particles are derived from the same or different hydrophobic material and each hydrophobic particle has a melting point below the melting point of the respective hydrophobic material, and further wherein the nanoemulsion further includes one or more pharmaceutically active agents and/or one or more chemiluminescent disease-detecting systems.

2. The topical composition of claim 1, wherein the hydrophobic particles have a melting point of from about 21° C. to about 33° C.

3. The topical composition of claim 2, wherein the hydrophobic material is selected from the group consisting of a wax, butter and combinations thereof.

4. The topical composition of claim 2, wherein the hydrophobic material is selected from the group consisting paraffin wax, beeswax white, candellila wax, mango butter, aloe butter, olive butter, coffea butter, macadamia nut butter, avocado butter, cocoa butter, seed hemp seed butter, illipe seed butter, kokum seed butter, pistachio nut butter, shea butter, sweet almond butter, grape seed butter, mowrah butter, apricot butter, sal butter, soy butter, wheat germ butter, palm kernel flakes and mixtures thereof.

5. The topical composition of claim 2, wherein the hydrophobic particles are from about 5 to about 50 nm in size.

6. The topical composition of claim 1, wherein the hydrophilic coating comprises a surfactant.

7. The topical composition of claim 6, wherein the surfactant is a non-ionic surfactant selected from the group consisting of a fatty alcohol, a polyoxyalkylene alkyl ether, a polyoxyalkylene alkenyl ether, a polyoxyethylene glycol octylphenol ether, a polyethylene glycol alkyl ether, a polyglyceryl fatty acid ester, a fatty acid mono- or diethanolamide, a polyoxyethylene hydrogenated castor oil, a polyoxyethylene sorbitan fatty acid ester, a polyoxyethylene fatty acid ester, a sorbitan ester, a sucrose ester, an alkyl-saccharide-based surfactant, an alkylamine oxide, an alkylamidoamine oxide and mixtures thereof.

8. The topical composition of claim 1, wherein the one or more chemiluminescent disease-detecting systems include a peroxide-reactive compound and a light-emitting material.

9. The topical composition of claim 8, wherein the peroxide-reactive compound is a compound of the formula: 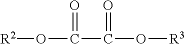
wherein R² and R³ are independently a substituted or unsubstituted aryl group, or a substituted or unsubstituted heteroaryl.

10. The topical composition of claim 8, wherein the peroxide-reactive compound is selected from the group consisting of an iptycene compound, bis(2-carbopentyloxy-3,5,6-trichlorophenyl oxalate, bis(2-nitrophenyl) oxalate, bis(2,4-dinitrophenyl) oxalate, bis(2,6-dichloro-4-nitrophenyl) oxalate, bis(2,4,6-trichlorophenyl) oxalate, bis(3-trifluoromethyl-4-nitrophenyl) oxalate, bis(2-methyl-4,6-dinitrophenyl) oxalate, bis(1,2-dimethyl-4,6-dinitrophenyl) oxalate, bis(2,4-dichlorophenyl) oxalate, bis(2,5-dinitrophenyl) oxalate, bis(2-formyl-4-nitrophenyl) oxalate, bis(pentachlorophenyl) oxalate, bis(pentafluorophenyl) oxalate, bis(1,2-dihydro-2-oxo-1-pyridyl) glyoxal, bis-N-phthalmidyl oxalate, bis(6-carbopentoxy-2,4,5-trichlorophenyl)oxalate (CPPO), bis(2,4,5-trichloro-6-carbobutoxyphenyl) oxalate, bis(2,4,6-trichlorophenyl)oxalate (TCPO), bis(2,4,5-trichloro-6-carbopentoxyphenyl) oxalate, bis(2,4,5-trichloro-6-carbobutoxyphenyl) oxalate, bis(2,4,6-trichlorophenyl) oxalate, 1,1-oxalyldiimidazole and phthalimido 3,6,6-trisulfo-2-naphthyl oxalate.

11. The topical composition of claim 8, wherein the light-emitting material has an emission spectrum between about 400 to about 800 nm.

12. The topical composition of claim 8, wherein the light-emitting material is anthracene, diphenylanthracene, or 9,10-bis(phenylethynyl)anthracene.

13. The topical composition of claim 8, wherein the one or more chemiluminescent disease-detecting systems further include a source of energy capable of converting an enzyme that catalyzes an oxidation-reduction reaction to hydrogen peroxide.

14. The topical composition of claim 13, wherein the source of energy is selected from the group consisting of malate oxidase, hexose oxidase, glucose oxidase, glutamate oxidase, cholesterol oxidase, aryl-alcohol oxidase, L-gulonolactone oxidase, galactose oxidase, pyranose oxidase, L-sorbose oxidase, pyridoxine 4-oxidase, alcohol oxidase, catechol oxidase, (S)-2-hydroxy-acid oxidase, ecdysone oxidase, choline oxidase, secondary-alcohol oxidase, 4-hydroxymandelate oxidase, long-chain-alcohol oxidase, glycerol-3-phosphate oxidase, thiamine oxidase, hydroxyphytanate oxidase, nucleoside oxidase, N-acylhexosamine oxidase, polyvinyl-alcohol oxidase, D-arabinono-1,4-lactone oxidase, vanillyl-alcohol oxidase, nucleoside oxidase, D-mannitol oxidase, xylitol oxidase, monoamine oxidase, xanthine oxidase such as hypoxanthine, L-gulonolactone oxidase, and lysyl oxidase and mixtures thereof.

15. The topical composition of claim 1, further comprising further contain one or more pharmaceutically acceptable excipients or carriers.

16. The topical composition of claim 15, wherein the pharmaceutically acceptable excipients or carriers include one or more of thickening agents, pigments, preservatives, pH adjusting agents, hydrating agents, and utraviolet-absorbing agents.

17. The topical composition of claim 1, which is in a semisolid form.

18. The topical composition of claim 1, which is a gel, cream, lotion, suspension, emulsion, ointment, foam, or paste.

19. A method for detecting a disease or a condition in need of treatment, the method comprising (a) contacting an area of a host with an effective amount of a topical composition comprising a nanoemulsion of a plurality of hydrophobic particles having a hydrophilic coating therein, wherein the hydrophobic particles are derived from the same or different hydrophobic material and has a melting point below the melting point of the respective hydrophobic material, and further wherein the nanoemulsion further includes one or more chemiluminescent disease-detecting systems; and (b) determining if the one or more chemiluminescent disease-detecting system emits a detectable chemiluminescent thereby indicating the presence of a disease or a condition.

20. The method of claim 19, wherein the one or more chemiluminescent disease-detecting systems include a peroxide-reactive compound and a light-emitting material.

21. The method of claim 20, wherein the peroxide-reactive compound is a compound of the formula: 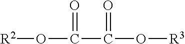
wherein R² and R³ are independently a substituted or unsubstituted aryl group, or a substituted or unsubstituted heteroaryl.

22. The method of claim 20, wherein the peroxide-reactive compound is selected from the group consisting of an iptycene compound, bis(2-carbopentyloxy-3,5,6-trichlorophenyl oxalate, bis(2-nitrophenyl) oxalate, bis(2,4-dinitrophenyl) oxalate, bis(2,6-dichloro-4-nitrophenyl) oxalate, bis(2,4,6-trichlorophenyl) oxalate, bis(3-trifluoromethyl-4-nitrophenyl) oxalate, bis(2-methyl-4,6-dinitrophenyl) oxalate, bis(1,2-dimethyl-4,6-dinitrophenyl) oxalate, bis(2,4-dichlorophenyl) oxalate, bis(2,5-dinitrophenyl) oxalate, bis(2-formyl-4-nitrophenyl) oxalate, bis(pentachlorophenyl) oxalate, bis(pentafluorophenyl) oxalate, bis(1,2-dihydro-2-oxo-1-pyridyl) glyoxal, bis-N-phthalmidyl oxalate, bis(6-carbopentoxy-2,4,5-trichlorophenyl)oxalate (CPPO), bis(2,4,5-trichloro-6-carbobutoxyphenyl) oxalate, bis(2,4,6-trichlorophenyl)oxalate (TCPO), bis(2,4,5-trichloro-6-carbopentoxyphenyl) oxalate, bis(2,4,5-trichloro-6-carbobutoxyphenyl) oxalate, bis(2,4,6-trichlorophenyl) oxalate, and phthalimido 3,6,6-trisulfo-2-naphthyl oxalate.

23. The method of claim 20, wherein the light-emitting material has an emission spectrum between about 400 to about 800 nm.

24. The method of claim 20, wherein the light-emitting material is anthracene, diphenylanthracene, or 9,10-bis(phenylethynyl)anthracene.

25. The method of claim 19, wherein the one or more chemiluminescent disease-detecting systems further include a source of energy capable of converting an enzyme that catalyzes an oxidation-reduction reaction to hydrogen peroxide.

26. The method of claim 25, wherein the source of energy is selected from the group consisting of malate oxidase, hexose oxidase, glucose oxidase, glutamate oxidase, cholesterol oxidase, aryl-alcohol oxidase, L-gulonolactone oxidase, galactose oxidase, pyranose oxidase, L-sorbose oxidase, pyridoxine 4-oxidase, alcohol oxidase, catechol oxidase, (S)-2-hydroxy-acid oxidase, ecdysone oxidase, choline oxidase, secondary-alcohol oxidase, 4-hydroxymandelate oxidase, long-chain-alcohol oxidase, glycerol-3-phosphate oxidase, thiamine oxidase, hydroxyphytanate oxidase, nucleoside oxidase, N-acylhexosamine oxidase, polyvinyl-alcohol oxidase, D-arabinono-1,4-lactone oxidase, vanillyl-alcohol oxidase, nucleoside oxidase, D-mannitol oxidase, xylitol oxidase, monoamine oxidase, xanthine oxidase such as hypoxanthine, L-gulonolactone oxidase, and lysyl oxidase and mixtures thereof.

27. A method for treating a host for a disease condition, the method comprising topically applying an effective amount of the topical composition of claim 1 to the host.

28. A test kit for detecting a disease or a condition in a host, the kit comprising (a) a source of a topical composition comprising a nanoemulsion of a plurality of hydrophobic particles having a hydrophilic coating therein, wherein the hydrophobic particles are derived from the same or different hydrophobic material and has a melting point below the melting point of the respective hydrophobic material, and further wherein the nanoemulsion further includes one or more chemiluminescent disease-detecting systems; and (b) a light emitting detector for detecting a disease or a condition in the host.
